(19) United States
(12) Patent Application Publication
(10) Pub. No.: US 2019/0236840 A1
(43) Pub. Date: Aug. 1, 2019

(54) SYSTEM AND METHOD FOR PATIENT ENGAGEMENT

(71) Applicant: Surgical Theater LLC, Mayfield Village, OH (US)

(72) Inventors: Alon ZUCKERMAN, Marina Del Rey, CA (US); Alon Yakob Geri, Beachwood, OH (US); Mordechai Avisar, Highland Heights, OH (US)

(21) Appl. No.: 16/259,851

(22) Filed: Jan. 28, 2019

Related U.S. Application Data
(60) Provisional application No. 62/622,581, filed on Jan. 26, 2018.

(51) Int. Cl. G06T 19/00; G06F 17/50; G16H 70/20; G16H 30/20; G16H 10/60; G02B 27/01; G06F 3/01

(52) U.S. Cl. CPC G06T 19/003; G06F 17/5009; G16H 70/20; G06F 3/012; G16H 10/60; G02B 27/017; G16H 30/20

(57) ABSTRACT

A method includes: storing static medical images of the patient; generating a three-dimensional model of the patient's anatomy by converting the medical images of the patient into the three-dimensional model; storing the three-dimensional model; displaying to a user, on a first display, the three-dimensional model of the patient in an interactive manner; displaying to the patient, on a second display, the three-dimensional model contemporaneously displayed in an interactive manner on the first display; providing an input interface to the user, wherein the input interface is configured to accept from the user clarifying material representative of information about a medical procedure adapted for the patient; displaying the three-dimensional model on the first display in a manner modified with said clarifying material; and also displaying the three-dimensional model on the second display in the manner modified with said clarifying material.

### CROSS-REFERENCE TO RELATED APPLICATION

This application claims priority from U.S. provisional patent application Ser. No. 62/622,581 filed on Jan. 26, 2018 which is incorporated by reference herein in its entirety.

### FIELD OF DISCLOSURE

The present disclosure relates to the field of surgical procedures and more specifically to the field of patient engagement.

### BACKGROUND

When facing a complex surgical procedure, a patient may often experience fear and anxiety in the days and weeks leading up to the surgery. This may be the result of the patient not clearly understanding the procedure and therefore not knowing what to expect. Engaging the patient prior to the surgical procedure and educating the patient may help alleviate this fear and anxiety. Clearer communication between the treating physician and the patient pertaining the pathological situation of the patient and the proposed solution is important in overcoming uncertainties the patient may feel and in establishing trust between the patient on one hand, and the physician and the healthcare provider on the other. This is also important due to the competitive environment in which healthcare providers operate today, and the many options patients face in selecting physicians and providers. In addition, by engaging the patient and educating the patient about the procedure, the patient may be more likely to take appropriate care and steps to ensure a proper recovery without complications and without need for returning to the hospital for follow up care. Existing techniques for engaging and educating a patient, however, such as showing the patient an image of the anatomy or a 3D model, may not be effective, particularly when the surgery involves a part of the anatomy that is abstract or difficult to understand in the context of a standalone image or even a 3D model.

Moreover, it may be desirable for a surgeon or other medical professional to collect data with respect to the patient's engagement for the purpose satisfying hospital regulations. For example, a hospital may require a patient to provide some form of acknowledgement that they have been presented with information about the surgical procedure, that they understand the procedure, and that they consent to the surgical procedure. However, existing forms of documenting patient engagement may not be efficient or effective. Moreover, existing forms of documenting patient engagement may not provide for proper data with respect to ensuring that a patient truly understands every step of a complex surgical procedure, which may involve multiple steps as well as the risks associated with such procedure.

Finally, it may be beneficial to include multiple medical professionals when engaging with a patient for a consultation prior to the surgical procedure, some of whom may be located in remote locations with respect to the patient and/or the primary treating physician(s). Existing protocols and procedures for engaging patients may not enable or accommodate the inclusion of remotely located medical professionals in the process.

### SUMMARY

A system for engaging a patient using a simulation of the patient's anatomy includes a first display, a second display, and a computer including one or more processors, one or more computer-readable tangible storage devices, and program instructions stored on at least one of the one or more storage devices for execution by at least one of the one or more processors. The program instructions are configured to store static medical images of the patient in an image database; generate a three-dimensional model of the patient's anatomy by converting the medical images of the patient into the three-dimensional model; store the three-dimensional model of the anatomy of the patient in a model database; display to a user who is a provider of medical services, on the first display, the three-dimensional model of the patient in an interactive manner; display to the patient, on the second display, the three-dimensional model of the patient contemporaneously displayed in an interactive manner on the first display; provide an input interface to the user, wherein the input interface is configured to accept from the user clarifying material representative of information about a medical procedure adapted for the patient; in response to receiving the clarifying material from the user, display the three-dimensional model on the first display in a manner modified with said clarifying material; and in response to receiving the clarifying material from the user, also display the three-dimensional model on the second display in the manner modified with said clarifying material.

A method of using a simulation of a patient's anatomy for engaging the patient includes the steps of: storing static medical images of the patient in an image database; generating a three-dimensional model of the patient's anatomy by executing software on a computer system to convert the medical images of the patient into the three-dimensional model; storing the three-dimensional model of the anatomy of the patient in a model database; executing software on a computer system to display to a user who is a provider of medical services, on a first display, the three-dimensional model of the patient in an interactive manner; executing software on the computer system to display to the patient, on a second display, the three-dimensional model of the patient contemporaneously displayed in an interactive manner on the first display; executing software on the computer system to provide an input interface to the user, wherein the input interface is configured to accept from the user clarifying material representative of information about a medical procedure adapted for the patient; in response to receiving the clarifying material from the user, executing software on the computer system to display the three-dimensional model on the first display in a manner modified with said clarifying material; and in response to receiving the clarifying material from the user, executing software on the computer system to also display the three-dimensional model on the second display in the manner modified with said clarifying material.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example patient engagement system.

FIG. 2 illustrates an example augmented reality application of an example patient engagement system.

FIG. 3 illustrates an example patient engagement system.

FIG. 4 illustrates an example patient consent application of an example patient engagement system.

FIG. 5 illustrates an example patient engagement system.

FIG. 6 illustrates an example patient engagement system.

FIG. 7 illustrates an example electronic health record application of an example patient engagement system.

FIG. 8 illustrates a block diagram of an example patient engagement computer of an example patient engagement system.

FIG. 9 illustrates an example method engaging a patient.

FIG. 10 illustrates an example computer for implementing an example patient engagement computer.

### DETAILED DESCRIPTION

The following acronyms and definitions will aid in understanding the detailed description:

AR—Augmented Reality—A live view of a physical, real-world environment whose elements have been enhanced by computer generated sensory elements such as sound, video, or graphics.

VR—Virtual Reality—A 3Dimensional computer generated environment which can be explored and interacted with by a person in varying degrees.

HMD—Head Mounted Display refers to a headset which can be used in AR or VR environments. It may be wired or wireless. It may also include one or more add-ons such as headphones, microphone, HD camera, infrared camera, hand trackers, positional trackers etc.

Controller—A device which includes buttons and a direction controller. It may be wired or wireless. Examples of this device are Xbox gamepad, PlayStation gamepad, Oculus touch, etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

Avatar—An avatar represents a user inside the virtual environment.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

A surgery rehearsal and preparation tool previously described in U.S. Pat. No. 8,311,791, incorporated in this application by reference, has been developed to convert static CT and MRI medical images into dynamic and interactive multi-dimensional full spherical virtual reality, six (6) degrees of freedom models (“MD6DM”) that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional 2 dimensional patient medical scans, that gives spherical virtual reality 6 degrees of freedom (i.e. linear; x, y, z, and angular, yaw, pitch, roll) in the entire volumetric spherical virtual reality model.

The MD6DM is built from the patient's own data set of medical images including CT, MRI, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved, and can be appreciated using the MD6DM.

The algorithm of the MD6DM takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD6DM reverts it to a 3D model by representing a 360° view of each of those points from both the inside and outside.

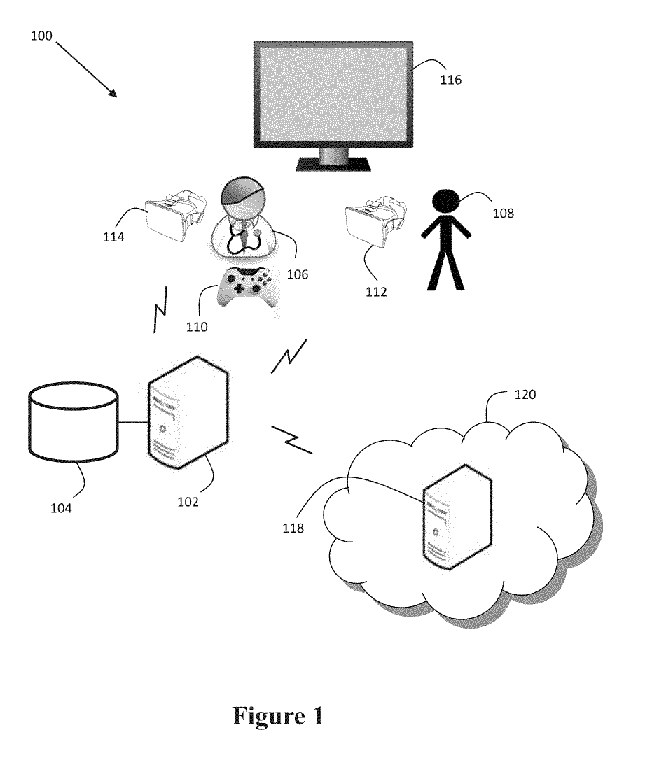

FIG. 1 illustrates an example patient engagement system 100 that leverages a prebuilt MD6DM model in order to effectively and efficiently engage the patient. In particular, the patient engagement system 100 enables a physician to educate the patient about a surgical procedure as well as to simultaneously and automatically document the patient's understanding of the surgical procedure, and the associated risks.

The patient engagement system 100 includes a patient engagement computer 102 that communicates with and receives a prebuilt MD6DM 360 model (not shown) from a MD6DM data store 104. In one example, the patient engagement computer 102 enables a physician 106, or other user or administrator, to modify the 360 model in advance of a consultation with a patient 108. For example, the physician may add notes or other suitable information to the 360 model that may be beneficial to have during the consultation or to show the patient 108.

The patient engagement computer 102 enables the physician 106 to engage the patient 108 by explaining a medical procedure to the patient 108 using the 360 virtual model, once it's loaded from the data store 104. In particular, the physician 106 interacts with the patient engagement computer 102 via a controller 110, or other suitable input device, and navigates into and around the 360 model, which is representative of the patient's 108 anatomy. In other words, the physician 106 virtually tours the inside of the patient's 108 body and is able to fly inside the body. A patient HMD 112 enables the patient 108 to follow along and view his/her own anatomy virtually, as represented by the 360 model, as the physician 106 navigates the 360 model. This is made possible by the patient engagement computer 102 receiving navigation control instructions from the controller 110 and translating those instructions in order to render appropriate images, which the patient engagement computer 102 communicates to the patient HMD 112 for viewing by the patient 108. This is accomplished in real time, based on input from the physician 106 via the controller 110.

The patient engagement system 100 further includes a physician HMD 114 to receive the same images as generated by the patient engagement computer 102 and communicated to the patient HMD 112. Thus, the physician 106 is provided with the same simultaneous virtual 360 view of the anatomy as represented by the 360 model as the patient 108 is viewing. The virtual view provided by the physician HMD 114 also enables the physician to accurately identify and navigate, using the controller 110, to locations within the 360 model for the purpose of engaging the patient 108. In other words, the patient HMD 112 mirrors the images and interactions of the physician HMD 114.

In one example, the patient engagement system 100 includes a display screen 116 to receive the same images as generated by the patient engagement computer 102 and communicated to the patient HMD 112. Thus, in one example, the physician 106 may view the 360 model on the display screen 116, instead of using a physician HMD 114, while the patient 108 views the 360 model via the patient HMD 112. In one example, both the physician 106 and the patient 108 may both view the anatomy as represented by the 360 model together simultaneously on the display screen 114.

As the physician 106 navigates the anatomy represented by the 360 model, the physician 106 engages the patient 108 by stopping to explain various points of interest as related to a surgical procedure to be performed. For example, the physician 106 may explain what the different parts of the anatomy are and how the surgical procedure will be performed in particular with reference to specific parts of the anatomy. The physician 106 may tour or navigate to different parts of the anatomy, or navigate to different sides and angles to gain different perspectives of the same part in the anatomy, in order to engage the patient 108 and to help the patient 108 better understand the surgical procedure to be performed. The physician 106 may stop to answer questions as appropriate or backtrack and navigate back to a location in the anatomy in order to further explain a part or location a second time.

In one example, the patient engagement computer 102 is able to receive data input from the controller 110 that is indicative of a physician's 106 note, marker, or other type of input to display within the 360 model for the patient 108 to view and to communicate the data representative of the desired note, marker, etc. to the patient HMD 112 for display. For example, a physician 108 may wish to highlight a specific point of reference in the anatomy or to make a note about where and how a surgical procedure will be performed with reference to a specific point in order to create a more engaging experience for the patient 108, as compared to the physician 106 simply speaking about the point of reference. In one example, such notes or other data input may be saved along with the 360 model in the MD6DM data store 104 for later reference. In one example, the notes or other data is saved in a remote data store 118 located in the cloud 120.

In one example, the physician's 106 note may include an audio explanation corresponding to a specific point of reference in the anatomy. Further, the audio explanation may include several points corresponding to several points of reference. Thus, the patient engagement computer 102 is able to synchronize audio, such as audio explanations prepared by the physician 106 or other suitable audio, with the 360 model such that the audio is presented to the patient 108 at a timing that matches the presentation of the corresponding point of reference in the anatomy.

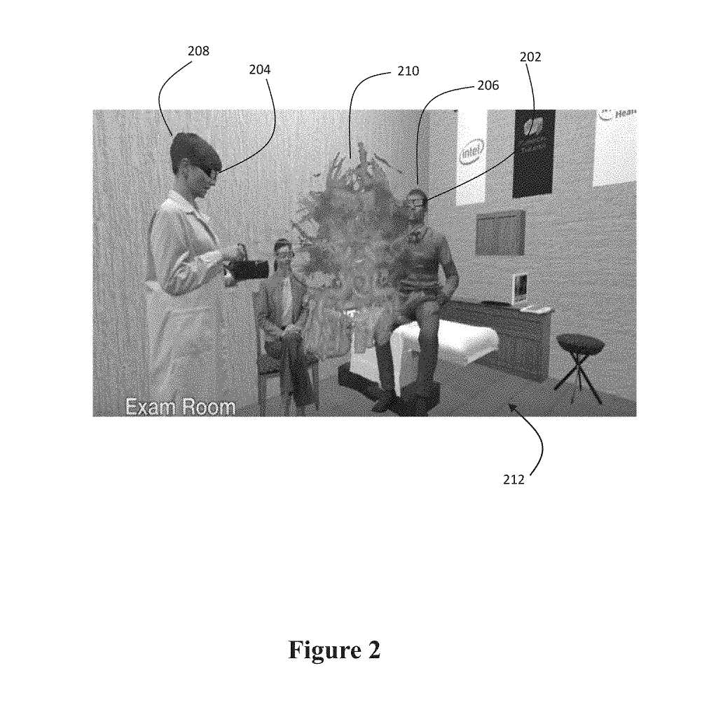

It should be appreciated that although the example patient engagement computer 102 is described as providing a virtual reality view to the patient 108 via the patient HMD 112, the patient engagement computer 102 may also provide, in one example, a mixed reality or augmented reality view to the patient 108 via the patient HMD 112. In such an example, as illustrated in FIG. 2, a patient augmented reality HMD 202 and a physician augmented reality HMD 204 may be semi-transparent so that the patient 206 may maintain eye contact with the physician 208 while the physician 208 educates the patient 206 about the surgical procedure, but at the same time be able to view the anatomy as represented by the 360 model 210 simultaneously in an augmented view inside the patient consultation room 212. Accordingly, the patient engagement computer 102 described in FIG. 1 may be configured to modify a virtual 360 model such that it is suited for augmented or mixed reality view. Moreover, the patient 206 and the physician may direct where and how the 360 model 210 is presented with respect to the patient consultation room 212.

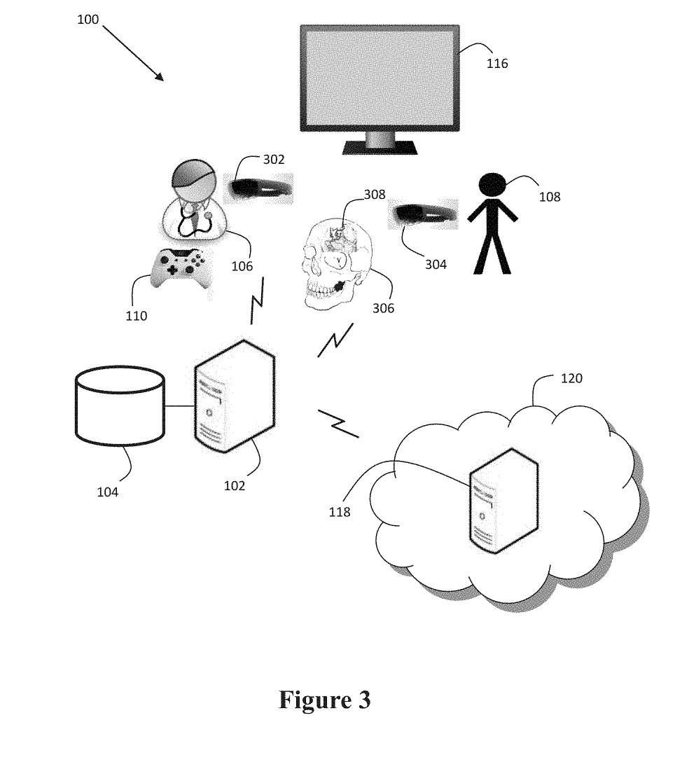

In one example, as illustrated in FIG. 3, the patient engagement system 100 includes physician augmented reality HMD 302 and a patient augmented reality HMD 304 to enable the physician 106 and patient 108 to view a physical model skull 306, or other suitable part of an anatomy, with a SNAP model 308 overlayed on top of the skull 406. In such an example, the patient engagement computer 102 receives tracking information indicating current location and angle of view of the physician 106 and the patient 108, respectively, generates a SNAP image in real time corresponding the determined location and angle of view, and communicates the generated image to the physician augmented reality HMD 302 and a patient augmented reality HMD 304, respectively. This enables the the physician 106 and patient 108 to examine a physical model skull 306 and see the anatomy inside as if they were looking at the inside of an actual brain. By leveraging tracking information, the physician 106 and the patient 108 are able to real time adjusted augmented reality views of the model skull 306 as they move around the model skull 306 and view it from different locations and angles. This further facilitates patient 108 engagement and understanding of the surgical procedure to be performed.

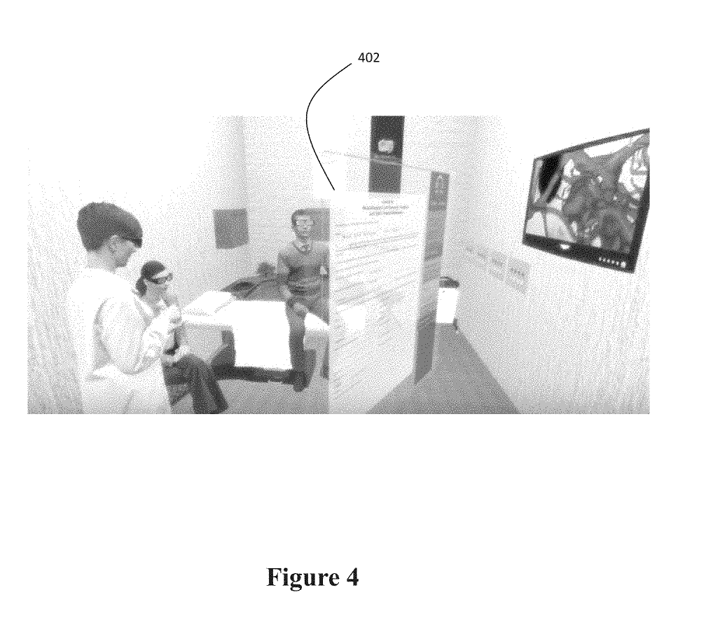

In one example, the patient engagement computer 102 receives and records data indicating a confirmation that the patient 108 received information about the surgical procedure form the physician 106 and that the patient 108 understands the procedure and the associated risks. For example, when the physician 106 and the patient 108 finish touring or flying through the anatomy and discussing the surgical procedure, the patient engagement computer 102 presents to the patient a digital form 402, as illustrated in FIG. 4, to sign, indicating that the patient 108 understand and consents to the procedure. The patient engagement computer 102 stores the signed consent form for later access as needed. In one example, the patient engagement computer 102 may enable the patient 108 to sign the consent form within the 360 virtual model via the patient HMD 112. For example, the patient 108 may sing the consent form by moving his head in such a way as to align a pointer within the 360 virtual model to point to a check box indicating consent.

In one example, the patient engagement computer 102 enables the physician 106 to stop and ask the patient 108 to give consent or to confirm understanding of the procedure multiple times during the consultation or fly through. For example, after a physician 106 navigates to a first position within the anatomy and explains a first step of a surgical procedure or a certain portion of a surgical procedure and associated risk if appropriate, the patient engagement computer 102 may provide the patient 108 with a virtual checkbox or some other suitable request for input which, upon receiving the input from the patient 108, confirms that the patient 108 understands and consents to that specific portion of information. The physician 106 may then proceed to explain additional information or steps of a surgical procedure, and the patient engagement computer 102 continues to records patient 108 consent as appropriate for each subsequent step or portion of information. Breaking down the consent process into multiple steps further facilitates patient 108 engagement by enabling the physician 106 to highlight certain specific portions of information and ensuring that the patient 108 understands each and every specific portion of information as appropriate.

It should be appreciated that the communication between HMDs, computers, and data stores as described herein may be facilitated either via wired or wireless connections. It should be further appreciated that, although the examples described herein illustrates a single patient 108 interacting with a single physician 106, the patient engagement computer 102 may facilitate engagement between multiple parties within the same room. For example, a patient's 108 friend or relative may join in and be similarly engaged via another HMD (not shown) or via the display screen 116. Similarly, more than one physician may join in on the consultation and engage with the patient 108. In such an example wherein multiple people engage in a consultation simultaneously, each party is represented by an individual avatar within 360 virtual model in order to facilitate interaction.

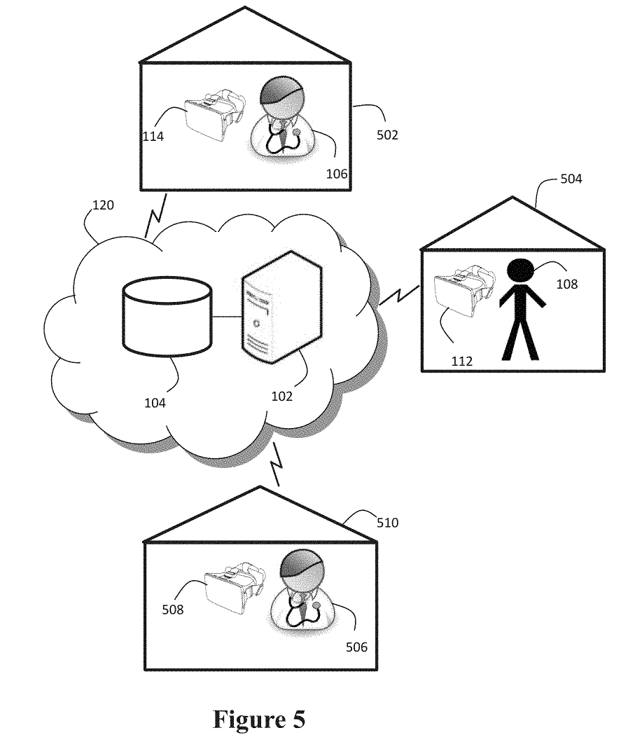

In one example, as illustrated in FIG. 5, one or more of the parties participating in the consultation may be located in a remote location. For example, the physician 106 may be physically located in a first hospital 502 while the patient participating in the consultation may be located at his home 504. A second physician 506 may participate in the same consultation, via a second physician HMD 508 in communication with the patient engagement computer 102 from a second hospital 510 remote from the first hospital 502. This enables multiple physicians of different levels of expertise and specialization to participate in a consultation simultaneously, thus further improving the patient's experience.

It should be appreciated that other suitable combinations of parties participating in a consultation from various locations may be facilitated by the patient engagement computer 102, other than the scenario illustrated in FIG. 5. For example, the patient 108 may be physically located in the same hospital room as the first physician 106 while interacting with a second physician 506 in a second remote hospital 510.

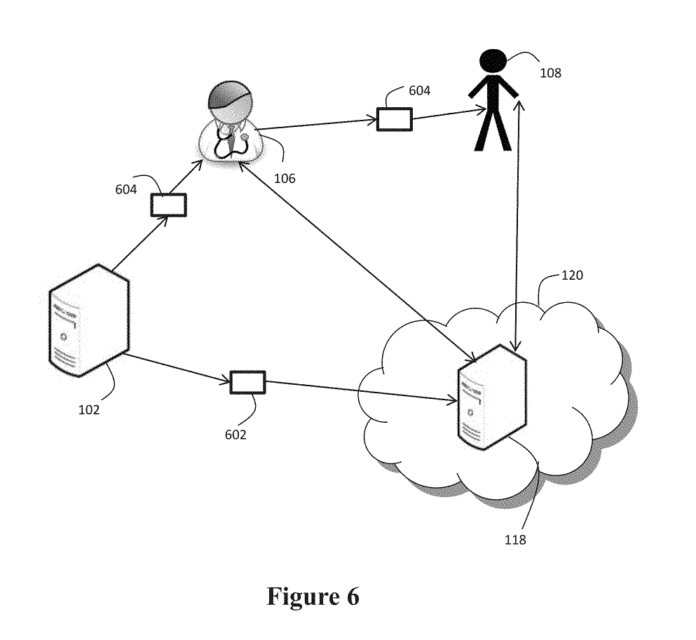

In one example, the patient engagement computer 102 may enable the patient 108 to sign the consent/acknowledgement as previously described, from a remote location such as a home 504. In one example, as illustrated in FIG. 6, the patient engagement computer 102 may enable the patient 108 to review the consultation at a later time at home or ant at any convenient time and location after the initial consultation with the physician 106, and to sign the consent/acknowledgement at any time after the initial consultation and before time of the actual surgical procedure. In particular, the patient engagement computer 102 automatically generates a recording of the consultation between the physician 106 and the patient 108, creating a patient engagement video 602 of the 360 virtual tour or fly through of the anatomy experienced during the consultation, including any suitable audio captured during the consultation between the physician 106 and the patient 108. The patient engagement video 602 may also include any suitable notes or additional information that the physician 106 added to the 360 model during the consultation or any other applicable interaction. The patient engagement computer 102 uploads the patient engagement video 602 to the remote data store 118 in the cloud 120.

The patient engagement computer 102 also creates a customized secure video link 604 for accessing the patient engagement video 602 at the remote data store 118. Thus, unauthorized access is prevented. The patient engagement computer 102 communicates the video link 604 to the physician 106. Once the physician 106 receives the link 604, the physician 106 may review and edit the patient engagement video 602 or add any additional notes as appropriate before sending the video link 604 to the patient 108.

It should be appreciated that the video link 604 may be communicated via any suitable means, such as by mail, email, text message, and so on. In one example, the patient engagement computer 102 may generate a printout for the patient 108, including the video link 604, with information about how to access the patient engagement video 602, or communicate the link to the patient 108 directly via other suitable means, immediately after the consultation. In such an example, access to the patient engagement video 602 may be locked until the physician 106 reviews the video, approves it, and grants access to the patient 108. This eliminates the extra step of the requiring the physician to forward the video link 604 to the patient 108.

It should be appreciated that once the patient engagement video 602 is uploaded to the remote data store 118, it can be access by the patient 108 any time from any location, as convenient for the patient 108 in order for the patient 108 to continue to be engaged after the initial consultation, up to the day of the actual surgical procedure. In one example, the patient engagement computer 102 may monitor how often and when patient 108 accesses the video 602 and communicate such information to the physician 106. Having such information may enable the physician 106 to better interact with and engage the patient 108 prior to the surgical procedure.

In one example, if the patient engagement computer 102 determines that the patient 108 has not accessed the patient engagement video 602 or has not completed a required consent form, the patient engagement computer 102 may send an alert or a reminder to either the patient 108 or the physician 106, or both, via suitable communication methods such as email, text message, phone call, and so on.

It should be appreciated that the patient engagement video 602 is stored at the remote data store 118 in compliance with HIPAA rules, or other applicable regulatory rules and regulations, and unauthorized access is prevented.

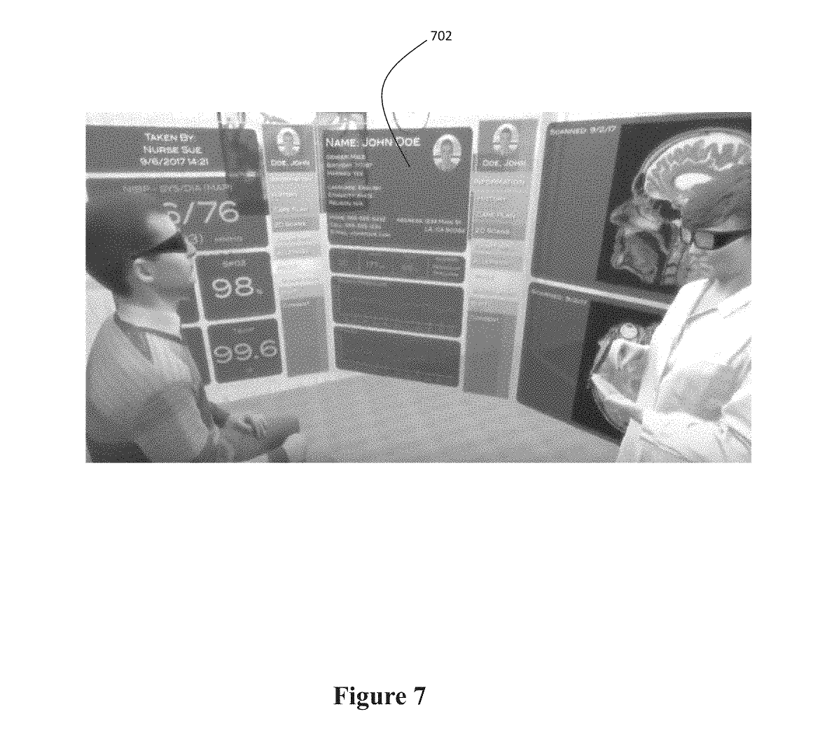

In one example, the patient engagement computer 102 integrates with additional data sources (not shown) such as Electronic Health Records (EHR) systems. In such an example, the patient engagement computer 102 is able to provide additional suitable content or information to the patient in a virtual or augmented manner, in addition to providing the 360 virtual model, thereby enabling further engagement. For example, as illustrated in FIG. 7, an EHR record may be retrieved, specific to a patient, and provided in a virtual or augmented view to the patient as an EHR view 702 via a HMD. This may further facilitate engagement between a physician and a patient and enable discussion with respect to specific information contained in a patient's health record while also discussing a surgical procedure to be performed in a single virtual or augmented environment without having to switch between different systems or displays.

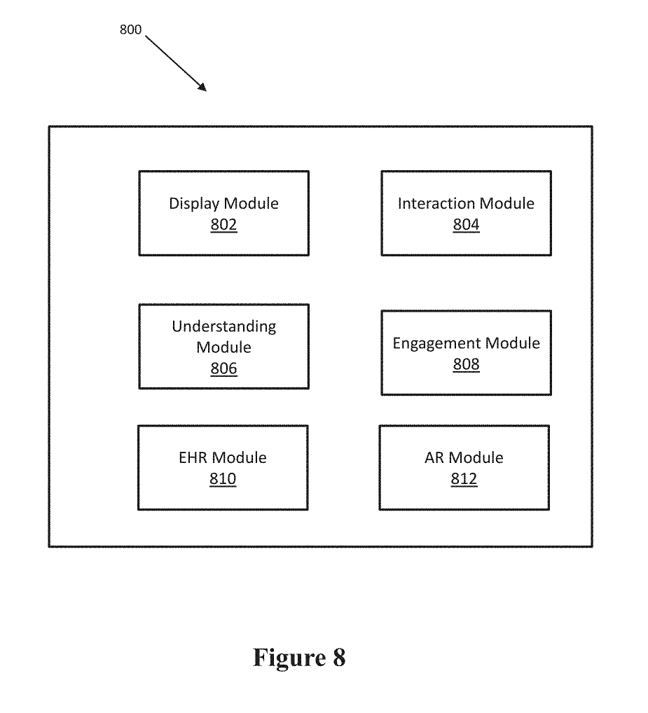

FIG. 8 illustrates a block diagram of an example patient engagement computer 800 (i.e. patient engagement computer 102 of FIG. 1). The patient engagement computer 800 includes a display module configured for providing an interactive three-dimensional model (i.e. a 360 virtual model previously described) representative of a patient's anatomy to a first display (not shown) such as an HMD.

The patient engagement computer 800 further includes an interaction module 804 configured for receiving data input indicative of an interaction with the three-dimensional model via the first display. The patient engagement computer 800 further includes an understanding module 806 configured for associating with the three-dimensional model a request for confirmation of the patient's understanding of the interaction. In one example, the interaction includes a simulation of movement along a path through the anatomy. In one example, the understanding module 806 is configured to associate with the three-dimensional model a plurality of requests for confirmation of the patient's understanding with a plurality positions within the path. In one example, the interaction includes associating an audio recording with a portion of the anatomy. In one example, the interaction includes associating a graphical note with a portion of the anatomy.

The patient engagement computer 800 further includes an engagement module 808 configured for engaging the patient. The engagement module 808 engages the patient by providing the interactive three-dimensional model to a second display such a second HMD, mirroring the interaction with the three-dimensional model on the second display, communicating the associated request for confirmation to the second display, and receiving data indicative of a confirmation of the patient's understanding of the interaction.

In one example, the engagement module 808 is further configured for generating a video representative of the interactive three-dimensional model, the mirrored interaction with the three-dimensional model, and the associated request for confirmation, and to communicate a link to the video. In one example, the engagement module 808 is further configured for activating the link, thereby granting access to the video. In one example, the engagement module 808 is further configured for monitoring an amount of time elapsed after communicating the link and communicating an alert indicative of a predetermined time elapsing without receiving the data indicative of the confirmation of the patient's understanding.

In one example, the patient engagement computer 800 further includes an electronic health records (“EHR”) module 810 configured for receiving an electronic health record associated with the patient and for providing the electronic health record to the first display. The EHR module 810 is further configured for receiving data input indicative of an interaction with the electronic health record, for providing the electronic health record to the second display, and for mirroring the interaction with the electronic health record on the second display.

In one example, the first and second displays are augmented reality head mounted displays. In such an example, an augmented reality (“AR”) module 812 is configured for receiving first tracking information indicating a first current location and angle of view of a first augmented reality head mounted display with respect to a first view of a physical object and for receiving second tracking information indicating a second current location and angle of view of a second augmented reality head mounted display with respect to a second view of the physical object. The AR module 812 is further configured for generating a first view of the three-dimensional model corresponding to the first tracking information, and for generating a second view of the three-dimensional model corresponding to the second tracking information. The AR module 812 is further configured for providing the first view of the three-dimensional model to the first augmented reality head mounted display, wherein the first view of the three-dimensional model aligns with and overlays the first view of the object, and for providing the second view of the three-dimensional model to the second augmented reality head mounted display, wherein the second view of the three-dimensional model aligns with and overlays the second view of the object.

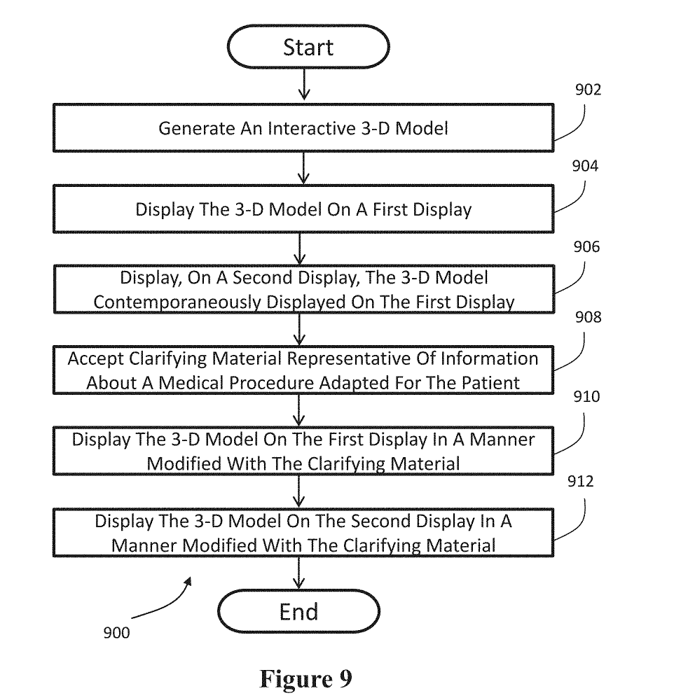

FIG. 9 is a flow chart illustrating an example method 900 for engaging a patient. At step 902, software executing on a computer system generates a three-dimensional model of the patient's anatomy by converting stored medical images of the patient into the three-dimensional model and stores the three-dimensional model of the anatomy of the patient in a model database. At step 904, software executing on a computer system displays to a user who is a provider of medical services, on a first display, the three-dimensional model of the patient in an interactive manner. At 906, software executing on a computer system displays to the patient, on a second display, the three-dimensional model of the patient contemporaneously displayed in an interactive manner on the first display. At 908, software executing on a computer system provides an input interface to the user, wherein the input interface is configured to accept from the user clarifying material representative of information about a medical procedure adapted for the patient. At 910, in response to receiving the clarifying material from the user, software executing on a computer system displays the three-dimensional model on the first display in a manner modified with said clarifying material. At 912, in response to receiving the clarifying material from the user, software executing on a computer system also displays the three-dimensional model on the second display in the manner modified with said clarifying material.

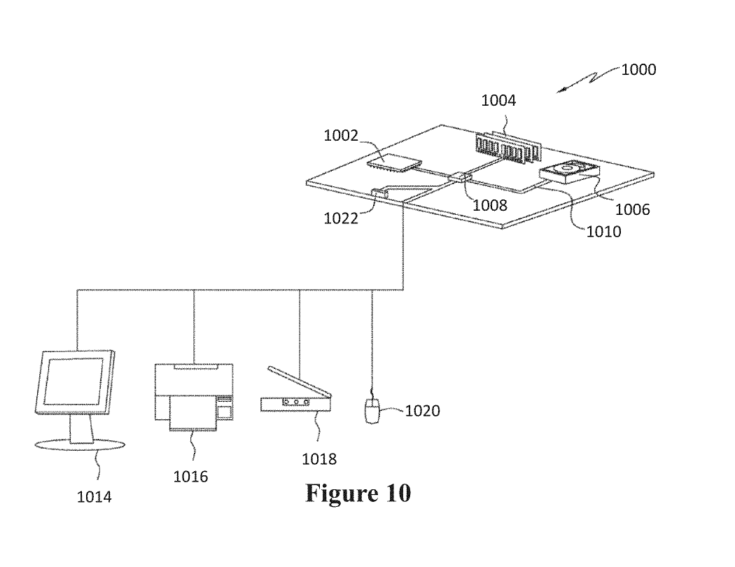

FIG. 10 is a schematic diagram of an example computer for implementing the example patient engagement computer 102 of FIGS. 1-8. The example computer 1000 is intended to represent various forms of digital computers, including laptops, desktops, handheld computers, tablet computers, smartphones, servers, and other similar types of computing devices. Computer 1000 includes a processor 1002, memory 1004, a storage device 1006, and a communication port 1008, operably connected by an interface 1010 via a bus 1012.

Processor 1002 processes instructions, via memory 1004, for execution within computer 800. In an example embodiment, multiple processors along with multiple memories may be used.

Memory 1004 may be volatile memory or non-volatile memory. Memory 1004 may be a computer-readable medium, such as a magnetic disk or optical disk. Storage device 1006 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 1004 or storage device 1006.

Computer 1000 can be coupled to one or more input and output devices such as a display 1014, a printer 1016, a scanner 1018, and a mouse 1020.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

1. A system for engaging a patient using a simulation of the patient's anatomy, the system comprising:
a first display;
a second display; and
a computer comprising one or more processors, one or more computer-readable tangible storage devices, and program instructions stored on at least one of the one or more storage devices for execution by at least one of the one or more processors, the program instructions being configured to:
store static medical images of the patient in an image database;
generate a three-dimensional model of the patient's anatomy by converting the medical images of the patient into the three-dimensional model;
store the three-dimensional model of the anatomy of the patient in a model database;
display to a user who is a provider of medical services, on the first display, the three-dimensional model of the patient in an interactive manner;
display to the patient, on the second display, the three-dimensional model of the patient contemporaneously displayed in an interactive manner on the first display;
provide an input interface to the user, wherein the input interface is configured to accept from the user clarifying material representative of information about a medical procedure adapted for the patient;
in response to receiving the clarifying material from the user, display the three-dimensional model on the first display in a manner modified with said clarifying material; and
in response to receiving the clarifying material from the user, also display the three-dimensional model on the second display in the manner modified with said clarifying material.

2. The system of claim 1, wherein the program instructions are further configured to provide a second input interface to the patient, wherein the second input interface is configured to accept from the patient data representative of the patient's understanding of and consent to the medical procedure adapted for the patient.

3. The system of claim 1, wherein the clarifying material comprises a simulation of movement along a path through the patient's anatomy.

4. The system of claim 1, wherein the clarifying material comprises at least one of an audio recording and a graphical notation.

5. The system of claim 1, wherein the program instructions are further configured to:
provide an electronic health record (“EHR”) interface configured to receive from an EHR system an EHR associated with the patient;
in response to receiving the EHR, to display the three-dimensional model on the first display in a manner modified with said EHR; and
in response to receiving the EHR, also display the three-dimensional model on the second display in the manner modified with said HER.

6. The system of claim, 1 wherein the first display comprises a first head mounted display and wherein the second display comprises a second head mounted display.

7. The system of claim 6, wherein the first head mounted display comprises a first augmented reality head mounted display and wherein the second head mounted display comprises a second augmented reality head mounted display.

8. The system of claim 7, wherein the program instructions are further configured to:
receive first tracking information indicating a first current location and angle of view of the first augmented reality head mounted display with respect to a first view of a physical object and receiving second tracking information indicating a second current location and angle of view of the second augmented reality head mounted display with respect to a second view of the physical object;
generate a first view of the three-dimensional model corresponding to the first tracking information, and generating a second view of the three-dimensional model corresponding to the second tracking information; and
provide the first view of the three-dimensional model to the first augmented reality head mounted display, wherein the first view of the three-dimensional model aligns with and overlays the first view of the object, and providing the second view of the three-dimensional model to the second augmented reality head mounted display, wherein the second view of the three-dimensional model aligns with and overlays the second view of the object.

9. The system of claim 2, wherein the program instructions are further configured to:
generate a video representative of the three-dimensional model as modified with the clarifying material, and as further modified with a request by the second input interface for the data representative of the patient's understanding of and consent to the medical procedure adapted for the patient;
store the video; and
communicate a link to the video.

10. The system of claim 9, wherein the program instructions are further configured to:
monitor an amount of time elapsed after communicating the link; and
communicate an alert indicative of a predetermined time elapsing without receiving from the patient the data representative of the patient's understanding of and consent to the medical procedure adapted for the patient.

11. A method of using a simulation of a patient's anatomy for engaging the patient, comprising the steps of:
storing static medical images of the patient in an image database;
generating a three-dimensional model of the patient's anatomy by executing software on a computer system to convert the medical images of the patient into the three-dimensional model;
storing the three-dimensional model of the anatomy of the patient in a model database;
executing software on a computer system to display to a user who is a provider of medical services, on a first display, the three-dimensional model of the patient in an interactive manner;
executing software on the computer system to display to the patient, on a second display, the three-dimensional model of the patient contemporaneously displayed in an interactive manner on the first display;
executing software on the computer system to provide an input interface to the user, wherein the input interface is configured to accept from the user clarifying material representative of information about a medical procedure adapted for the patient;
in response to receiving the clarifying material from the user, executing software on the computer system to display the three-dimensional model on the first display in a manner modified with said clarifying material; and
in response to receiving the clarifying material from the user, executing software on the computer system to also display the three-dimensional model on the second display in the manner modified with said clarifying material.

12. The method of claim 11, further comprising the steps of executing software on the computer system to provide a second input interface to the patient, wherein the second input interface is configured to accept from the patient data representative of the patient's understanding of and consent to the medical procedure adapted for the patient.

13. The method of claim 11, wherein the clarifying material comprises a simulation of movement along a path through the patient's anatomy.

14. The method of claim 11, wherein the clarifying material comprises at least one of an audio recording and a graphical notation.

15. The method of claim 11, further comprising the steps of:
executing software on the computer system to provide an electronic health record (“EHR”) interface configured to receive from an EHR system an EHR associated with the patient;
in response to receiving the EHR, executing software on the computer system to display the three-dimensional model on the first display in a manner modified with said EHR; and
in response to receiving the EHR, executing software on the computer system to also display the three-dimensional model on the second display in the manner modified with said EHR.

16. The method of claim 11, wherein the first display comprises a first head mounted display and wherein the second display comprises a second head mounted display.

17. The method of claim 16, wherein the first head mounted display comprises a first augmented reality head mounted display and wherein the second head mounted display comprises a second augmented reality head mounted display.

18. The method of claim 17, further comprising the steps of:
executing software on the computer system to receive first tracking information indicating a first current location and angle of view of the first augmented reality head mounted display with respect to a first view of a physical object and receiving second tracking information indicating a second current location and angle of view of the second augmented reality head mounted display with respect to a second view of the physical object;
executing software on the computer system to generate a first view of the three-dimensional model corresponding to the first tracking information, and generating a second view of the three-dimensional model corresponding to the second tracking information; and
executing software on the computer system to provide the first view of the three-dimensional model to the first augmented reality head mounted display, wherein the first view of the three-dimensional model aligns with and overlays the first view of the object, and providing the second view of the three-dimensional model to the second augmented reality head mounted display, wherein the second view of the three-dimensional model aligns with and overlays the second view of the object.

19. The method of claim 12, further comprising the step of:
executing software on the computer system to generate a video representative of the three-dimensional model as modified with the clarifying material, and as further modified with a request by the second input interface for the data representative of the patient's understanding of and consent to the medical procedure adapted for the patient;
executing software on the computer system to store the video; and
executing software on the computer system to communicate a link to the video.

20. The method of claim 19, further comprising the step of:
executing software on the computer system to monitor an amount of time elapsed after communicating the link; and
executing software on the computer system to communicate an alert indicative of a predetermined time elapsing without receiving from the patient the data representative of the patient's understanding of and consent to the medical procedure adapted for the patient.
